(19) United States
(12) Patent Application Publication
(10) Pub. No.: US 2006/0241456 A1
(43) Pub. Date: Oct. 26, 2006

(54) Ultrasonic imaging apparatus and ultrasonic imaging method

(75) Inventor: Hiroyuki Karasawa, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/338,781

(22) Filed: Jan. 25, 2006

(30) Foreign Application Priority Data
Feb. 8, 2005 (JP) 2005-031282

(51) Int. Cl. A61B 8/00

(52) U.S. Cl. 600/447

(57) ABSTRACT

An ultrasonic imaging apparatus capable of displaying an ultrasonic image clearly representing different tissues by discriminating ultrasonic echoes generated in regions having different reflection characteristics among the received ultrasonic echoes. The ultrasonic imaging apparatus includes: an ultrasonic probe including plural ultrasonic transducers for transmitting ultrasonic waves toward an object to be inspected and receiving ultrasonic echoes propagating from the object to output reception signals; a reflection signal evaluating unit for evaluating mutual property of a group of reception signals relating to a region within the object from among the reception signals respectively outputted from the plural ultrasonic transducers; and a variable amplifying unit for amplifying the group of reception signals with signal amplification factors determined with respect to respective reception signals based on an evaluation result of the reflection signal evaluating unit.

### BACKGROUND OF THE INVENTION

1. Field of the Invention

The present invention relates to an ultrasonic imaging apparatus and an ultrasonic imaging method for performing imaging of organs, bones, etc. within a living body by transmitting and receiving ultrasonic waves so as to generate ultrasonic images to be used for medical diagnosis.

2. Description of a Related Art

In an ultrasonic imaging apparatus to be used for medical diagnoses, an ultrasonic probe including plural ultrasonic transducers having transmitting and receiving functions of ultrasonic waves is used. When an ultrasonic beam formed by synthesizing plural ultrasonic waves is transmitted from such an ultrasonic probe to an object to be inspected, the ultrasonic beam is reflected at a boundary between regions having different acoustic impedances, i.e., between tissues within the object. Thus generated ultrasonic echoes are received and an image is constructed based on the intensity of the ultrasonic echoes, and thereby, the state within the object can be reproduced on a screen.

The intensity of the ultrasonic waves transmitted from the ultrasonic transducers is reduced according to the depth within the object due to the influence of ultrasonic energy absorption, refraction and scattering of ultrasonic beams, etc. in the object. Accordingly, the intensity of ultrasonic echoes received by the ultrasonic transducers attenuates according to the depth of reflection position. In order to correct such attenuation of ultrasonic echo intensity, a technique for changing the gain of an amplifier in the reception circuit according to time required from transmission of ultrasonic waves and reception of ultrasonic echoes (according to the depth of reflection position) has been conventionally used. Such technique is called STC (sensitivity time gain control) or TGC (time gain compensation).

However, in the case where there is a boundary having large reflectance in an ultrasonic wave transmission region, the intensity of ultrasonic echoes reflected at the boundary becomes extremely large. On this account, the boundary in the ultrasonic image generated by STC is displayed with high brightness, and the visibility of the image near the boundary becomes poor. For example, in an ultrasonic image obtained by ultrasonic imaging of a human body as shown in FIG. 18, the amplitude of ultrasonic echo signal reflected at a boundary between a soft tissue such as a muscle and a hard tissue such as a bone part becomes very large as shown in FIG. 19. Accordingly, the boundary between the bone part and the soft tissue in front thereof is displayed with high brightness. On the other hand, since great reflection occurs in the bone part, ultrasonic echoes from the interior of the bone part and rear part of the bone part become very weak. Furthermore, the influence of ringing due to ultrasonic echoes having high intensity remains until the time corresponding to the reception of ultrasonic echoes generated in the bone interior, and therefore, large-amplitude ringing will be added to the reception signals from the bone interior. However, it is generally impossible to separate weak signal components representing information of the bone interior from the reception signals to which ringing has been added. Further, regarding the ultrasonic echoes from the soft tissue present in front of the bone part, the visibility in the display screen is significantly deteriorated due to the presence of the ultrasonic echoes having large intensity generated on the surface of the bone part.

Thus, the ultrasonic echoes generated on the periphery of the hard tissue is buried in the ultrasonic echoes having large intensity generated in the hard tissue, and therefore, it is extremely difficult to clearly imaging the proximity to the hard tissue with high reflectance.

As a related technology, Japanese Patent Application Publication JP-A-7-236637 discloses an ultrasonic diagnostic apparatus for automatically controlling a gain of a reception analog circuit or a TGC gain to be kept properly. The ultrasonic diagnostic apparatus includes an ultrasonic probe for receiving ultrasonic waves and outputting ultrasonic echoes, a reception analog circuit for amplifying and analog processing the ultrasonic echoes and outputting sound ray signals, frame data generating means for generating frame data from the sound ray signals, and image display means for displaying images based on the frame data, and further includes representative value acquiring means for acquiring a representative value of the frame data and control signal output means for outputting control signals for changing the gain of the reception analog circuit based on the representative value (page 2).

According to JP-A-7-236637, an image is divided into plural partial areas, a representative value of frame data corresponding each partial areas is acquired, the representative value is monitored and fed back to corresponding TGC gain, and thereby, the gain in each partial region can be automatically and precisely maintained (page 4). However, the art disclosed in JP-A-7-236637 is to improve the image quality of an entire ultrasonic image, but not to improve the image quality of the image representing the region near the tissue with high reflectance such as a bone part.

Further, Japanese Patent Application Publication JP-A-7-323032 discloses an ultrasonic diagnostic apparatus for automatically performing accurate STC correction and constantly obtaining optimal tomographic images even in the case where conditions of an ultrasonic probe, a part to be diagnosed, an object to be inspected, etc. are changed. In the ultrasonic diagnostic apparatus, an STC circuit is formed by in addition to a gain control circuit, a smoothing circuit, a differentiating circuit, a threshold setting circuit, a first integrating circuit, a second A/D converter, a second integrating circuit and a second D/A converter (pages 1, 5 and 6, FIG. 1). Thereby, an STC curve that does not extremely amplify the echo-free part, nor extremely reduce the gain for a part to be displayed specifically brighter than the periphery such as a tumor existing in a tissue and make it difficult to discriminate the part from the peripheral tissue. However, the art disclosed in JP-A-7-323032 is also to improve the image quality of an entire ultrasonic image, but the improvement in the image quality of the image representing the region near the tissue with high reflectance cannot be expected.

By the way, when an ultrasonic image is generated, the use of elements other than intensity of ultrasonic echoes has been studied. It is conceivable that statistical property (statistics values) representing interrelationships among plural ultrasonic echoes respectively received by plural ultrasonic transducers are utilized as the elements.

As a related technology, Japanese Patent Application Publication JP-A-11-235341 discloses an ultrasonic diagnostic apparatus for suppressing the influence of distortion on image quality even when the waveform of reception signals is distorted due to refraction, multiple reflection or the like. The ultrasonic diagnostic apparatus is to obtain ultrasonic images by providing transmission and reception directivity to ultrasonic waves by providing individual delay times to respective excitation of arranged plural vibrators and reception signals obtained by these vibrators receiving ultrasonic reflection waves from an object to be inspected and scanning the interior of the object with the ultrasonic waves provided with directivity. The apparatus includes a reception signal evaluating unit for evaluating the distortion of reception signals with respect to each vibrator and an aperture control unit for controlling at least one of the intensity of the excitation signals and the amplification factor of the reception signals according to the evaluation result thereof. Further, the reception signal evaluating unit evaluates the degree of distortion of reception signals by utilizing the waveform similarity, correlation coefficient, intensity, etc. of the reception signals (pages 1 and 2).

That is, in JP-A-11-235341, in order to reduce the influence of the reception signals that have been distorted by the acoustic non-uniformity within a living body, phase addition is performed after the intensity or power of the reception signals with great distortion is reduced. Thereby, the improvement in image quality of the entire B-mode image can be expected. However, in JP-A-11-235341, the correlation of reception signals between vibrators is obtained only for obtaining the similarity of the reception signals for evaluate the distortion of reception signals, but the tissue property within the object are not obtained or a specific tissue is not extracted based on the relationship between reception signals.

Further, International Publication WO2001/80714 discloses an adaptive mapping method in a medical ultrasonic imaging system operative to acquire a reception input signal to display an output signal, and the adaptive mapping method includes the steps of: (a) determining a statistical measure of variability of the input signal; (b) identifying portions of the input signal corresponding to soft tissue based at least in part on the statistical measure at step (a); and (c) mapping the portions of the input signal identified at step (b) to a soft tissue range of output signal values. Further, in the method, a Rayleigh distribution as a spatial statistical distribution of amplitude of reflection signal is used for identifying the soft tissue.

In WO2001/80714, the object is to improve S/N of the signal representing the soft tissue, and the medical ultrasonic imaging system disclosed there has an automatic correction function for displaying the soft tissue in precise density. However, in the art disclosed in WO2001/80714, there is no viewpoint of extracting signals having small amplitude from signals having large amplitude, and therefore, an image representing the proximity to the hard tissue with high reflectance such as a bone part can not be displayed appropriately.

### SUMMARY OF THE INVENTION

The present invention has been achieved in view of the above-mentioned problems. An object of the present invention is to provide an ultrasonic imaging apparatus and an ultrasonic imaging method capable of displaying an ultrasonic image clearly representing different tissues by discriminating ultrasonic echoes generated in regions having different reflection characteristics among the received ultrasonic echoes. Specifically, an object of the present invention is to appropriately display the proximity to a tissue having a high reflectance by extracting signals having small amplitude buried in the signals having large amplitude.

In order to solve the above-mentioned problems, an ultrasonic imaging apparatus according to one aspect of the present invention includes: an ultrasonic probe including plural ultrasonic transducers for transmitting ultrasonic waves toward an object to be inspected and receiving ultrasonic echoes propagating from the object to output reception signals; evaluating means for evaluating mutual property of a group of reception signals relating to a region within the object from among the reception signals respectively outputted from the plural ultrasonic transducers; and variable amplifying means for amplifying the group of reception signals with signal amplification factors determined with respect to respective reception signals based on an evaluation result of the evaluating means.

Further, an ultrasonic imaging method according to one aspect of the present invention is a method of obtaining information for generating an ultrasonic image based on reception signals obtained by using an ultrasonic probe including plural ultrasonic transducers for transmitting ultrasonic waves toward an object to be inspected and receiving ultrasonic echoes propagating from the object to output reception signals, and the method includes the steps of: (a) evaluating mutual property of a group of reception signals relating to a region within the object from among the reception signals respectively outputted from the plural ultrasonic transducers; and (b) amplifying the group of reception signals with signal amplification factors determined with respect to respective reception signals based on an evaluation result at step (a).

Note that, in the present application, the signal amplification factor includes a value of “1” or less.

According to the present invention, the signal amplification factors of the group of reception signals are adjusted with respect to respective reception signals based on the mutual property of the group of reception signals representing ultrasonic echoes generated in a certain region, and therefore, signal components relating to certain tissue property contained in the group of reception signals can be extracted. Thereby, signals having small amplitude, which are often buried in signals having large amplitude, can be extracted. Accordingly, by performing phasing addition on the group of reception signals with thus adjusted signal amplification factors, a B-mode image clearly representing different tissues can be generated. That is, even in the case where a hard tissue exists nearby, a soft tissue can be clearly displayed in the image.

### BRIEF DESCRIPTION OF THE DRAWINGS

FIG. 1 is a block diagram showing a constitution of an ultrasonic imaging apparatus according to the first to third embodiments of the present invention;

FIGS. 2A to 2C show an intensity distribution of reception signals when an ultrasonic beam is transmitted toward a specular reflector and received;

FIGS. 3A to 3C show an intensity distribution of reception signals when an ultrasonic beam is transmitted toward a scattering reflector and received;

FIGS. 4A to 4D show an intensity distribution of reception signals when an ultrasonic beam is transmitted toward a region where a soft tissue exists near a hard tissue and received;

FIG. 5 is a diagram for explanation of the operation of a tissue-by-tissue phasing addition method determining unit shown in FIG. 1;

FIG. 6 shows a frequency distribution of a group of reception signals representing ultrasonic echoes reflected by a specular reflector and a scattering reflector;

FIG. 7 is a diagram for explanation of a method of determining whether or not an analysis region is a specular reflector;

FIG. 8A shows a reflection distribution corresponding to a specular reflector, and FIG. 8B shows a frequency corresponding to the reflection distribution shown in FIG. 8A;

FIG. 9A shows a reflection distribution corresponding to a scattering reflector with relatively small variations, and FIG. 9B shows a frequency corresponding to the reflection distribution shown in FIG. 9A;

FIG. 10A shows a reflection distribution corresponding to a scattering reflector with relatively large variations;

FIG. 10B shows a frequency corresponding to the reflection distribution shown in FIG. 10A;

FIG. 11 is a schematic diagram showing an ultrasonic image generated by the ultrasonic imaging apparatus according to the first embodiment of the present invention;

FIG. 12 shows a histogram corresponding to a spatial intensity distribution of reception signals;

FIG. 13 is a chart showing classified parameters of beta distribution;

FIGS. 14A to 14C show the cases where beta distributions become U-shaped;

FIGS. 15A to 15D show the cases where beta distributions become J-shaped;

FIG. 16 shows a reflection distribution in the case where the beta distribution becomes J-shaped;

FIGS. 17A to 17C show the cases where beta distributions become single-peaked;

FIG. 18 shows the state in which an ultrasonic beam is transmitted from an ultrasonic transducer array to a human body; and

FIG. 19 shows a detection signal of ultrasonic echoes reflected at a boundary between a soft tissue and a hard tissue.

### DESCRIPTION OF THE PREFERRED EMBODIMENTS

Hereinafter, preferred embodiments of the present invention will be described in detail by referring to the drawings. The same reference numbers are assigned to the same component elements and the description thereof will be omitted.

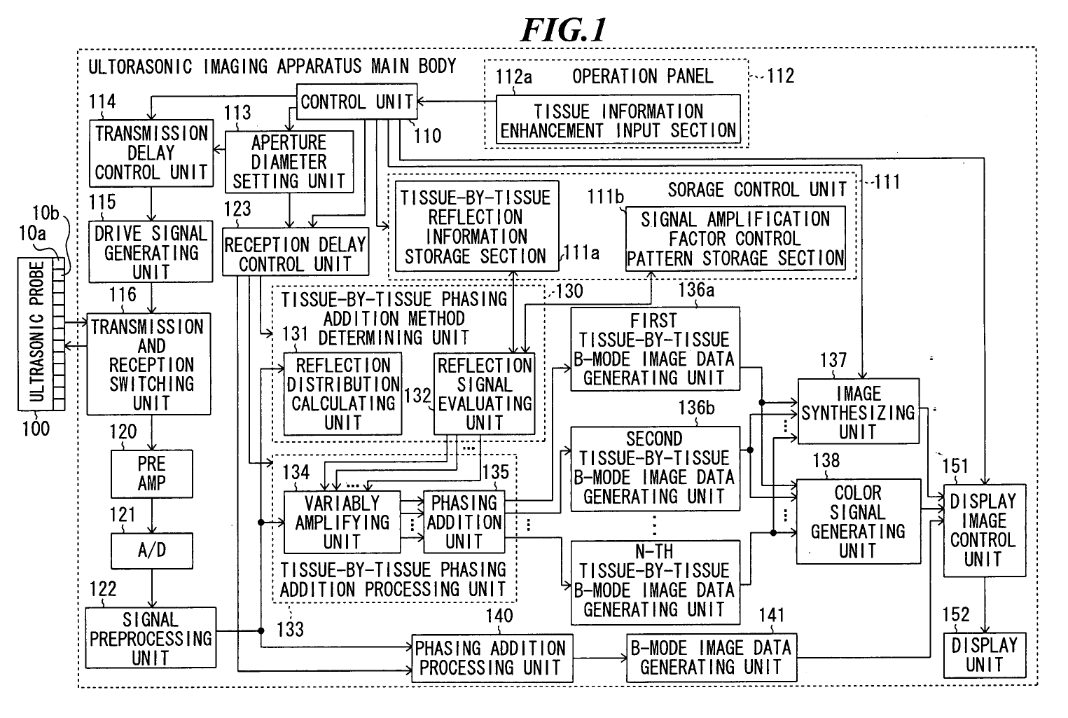

FIG. 1 is a block diagram showing a constitution of an ultrasonic imaging apparatus according to the first embodiment of the present invention. The ultrasonic imaging apparatus according to the embodiment includes an ultrasonic imaging apparatus main body and an ultrasonic probe 100 connected to the ultrasonic imaging apparatus main body by a cable.

The ultrasonic probe 100 is used by being abutted on an object to be inspected to transmit an ultrasonic beam to the object and receive ultrasonic echoes propagating from the object. The ultrasonic probe 100 includes plural ultrasonic transducers 10a, 10b, . . . for transmitting ultrasonic waves based on applied drive signals and receiving ultrasonic echoes to output reception signals. These ultrasonic transducers 10a, 10b, . . . are arranged in a one-dimensional or two-dimensional manner to form a transducer array.

Each ultrasonic transducer is constituted by a vibrator in which electrodes are formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride) or the like. When a voltage is applied to the electrodes of the vibrator by transmitting pulse electric signals or continuous wave electric signals, the piezoelectric material expands and contracts. By the expansion and contraction, pulse ultrasonic waves or continuous wave ultrasonic waves are generated from the respective vibrators, and an ultrasonic beam is formed by synthesizing these ultrasonic waves. Further, the respective vibrators expand and contract by receiving propagating ultrasonic waves and generate electric signals. These electric signals are output as reception signals (detection signals) of ultrasonic echoes.

Alternatively, as the ultrasonic transducers, plural kinds of elements of different conversion types may be used. For example, the above-mentioned vibrators are used as elements for transmitting ultrasonic waves and photo-detection type ultrasonic transducers are used as elements for receiving ultrasonic waves. The photo-detection type ultrasonic transducer is for detecting ultrasonic waves by converting ultrasonic signals into optical signals, and constituted by a Fabry-Perot resonator or fiber Bragg grating, for example.

Further, the ultrasonic imaging apparatus main body includes a control unit 110, a storage control unit 111, an operation panel 112, a transmission delay control unit 114, a drive signal generating unit 115, a transmission and reception switching unit 116, a preamplifier (PREAMP) 120, and an A/D converter 121, a signal preprocessing unit 122, a reception delay control unit 123, a tissue-by-tissue phasing addition method determining unit 130, a tissue-by-tissue phasing addition processing unit 133, first to N-th tissue-by-tissue B-mode image data generating units 136a, 136b, . . . , an image synthesizing unit 137, a color signal generating unit 138, a phasing addition processing unit 140, a B-mode image data generating unit 141, a display image control unit 151 and a display unit 152.

The control unit 110 controls each unit of the ultrasonic imaging apparatus according to the embodiment, and is formed by a CPU and software, for example.

The storage control unit 111 controls a recording medium for recording a fundamental program (software) for activating the CPU to execute operation, programs to be used f or performing various kinds of processing, and information to be used for those processing. As the recording medium, other than the built-in hard disk, an external hard disk, a flexible disk, an MO, an MT, a RAM, CD-ROM, DVD-ROM or the like may be used.

In the recording medium controlled by the storage control unit 111, a tissue-by-tissue reflection information storage section 111a and a signal amplification factor control pattern storage section 111b are formed as recording areas.

In the issue-by-tissue reflection information storage section 111a, plural kinds of tissue information associated with mutual property (also referred to as “reflection information”) of a group of reception signals representing ultrasonic echoes are stored. Here, the tissue information includes such tissue property that a target tissue is hard (e.g., a hard tissue such as a bone part, tendon or ligament) or soft (e.g., a soft tissue such as skin, muscle or blood vessel) and speckle patterns. Further, the mutual property of a group of reception signals includes a spatial intensity distribution of plural reception signals, statistics values obtained based thereon and so on.

The speckle pattern is a pattern in which bright parts and/or dark parts produced by interference between ultrasonic echoes are scattered, and seen in an ultrasonic image of an organ formed by many reflectors having sizes near the wavelength of ultrasonic waves such as a liver, for example. In the case where a tumor or the like is included in a tissue within an organ, but no clear reflection surface is seen at the outline of the tissue, sometimes the difference between a normal tissue and an abnormal tissue is determined by the difference between speckle patterns, and therefore, a speckle pattern is also an important element in medical diagnoses.

Further, in the signal amplification factor control pattern storage section 111b, a plurality of signal amplification factor control patterns (hereinafter, simply referred to as “amplification factor control patterns”) to be used for controlling signal amplification factors of the group of reception signals representing ultrasonic echoes generated within the object with respect to respective reception signals are stored in association with the plural kinds of tissue information. Alternatively, the plural amplification factor control patterns may be directly associated with the mutual property of the group of reception signals and stored. The mutual property of the group of reception signals, and relationship between the mutual property and the tissue information will be described later in detail.

The operation panel 112 includes a keyboard, adjustment knob, and a pointing device including a mouse or the like (e.g., a tissue information enhancement input section 112a) to be used when an operator inputs commands and information to the ultrasonic imaging apparatus.

An aperture diameter setting unit 113 sets the aperture diameter of the ultrasonic transducer array (i.e., plural ultrasonic transducers to be used) according to the transmission direction, reception direction, and depth of focus of an ultrasonic beam transmitted from the ultrasonic probe 100 so that a certain region within the object is scanned by the ultrasonic beam.

The transmission delay control unit 114 sets delay times to be provided to the plural ultrasonic transducers included in the aperture set in the aperture diameter setting unit 113.

The drive signal generating unit 115 includes plural drive circuits for generating plural drive signals to be supplied to the plural ultrasonic transducers, respectively. These drive circuits generate drive signals based on the delay times that have been set in the transmission delay control unit 114.

The transmission and reception switching unit 116 switches between a transmission mode in which drive signals are supplied to the ultrasonic probe 100 and a reception mode in which reception signals are outputted from the ultrasonic probe 100 under the control of the control unit 110.

The preamplifier 120 and the A/D converter 121 have plural channels corresponding to the plural ultrasonic transducers 10a, 10b, . . . , and input reception signals outputted from the plural ultrasonic transducers and perform preamplification and analog to digital conversion on the respective reception signals.

The signal preprocessing unit 122 performs the following intensity corrections (i) to (iii) according to need on the plural reception signals that have been A/D converted.

(i) Element Sensitivity Correction

Variations in performance of ultrasonic transducers generated when an ultrasonic transducer array is manufactured are corrected. The correction can be performed in the manner in which a correction table is created in advance by transmitting and receiving ultrasonic beams from the ultrasonic probe 100 using a standard reflection source and measuring the characteristics of the respective ultrasonic transducers, and the correction table is used at the time of processing of reception signals.

(ii) Solid Angle Intensity Correction

In an ultrasonic transducer array, since the solid angle relative to the reflection position of the ultrasonic echo becomes smaller, as an ultrasonic transducer is located closer to the end of the aperture, apparent reception intensity becomes smaller. Accordingly, intensity correction is performed on the reception signals according to the reception depth (the depth of the reflection point where the ultrasonic echo is generated), positional relationship with the respective ultrasonic transducers, and differences in reception solid angle between ultrasonic transducers determined by the aperture.

(iii) Distance Correction

The distance attenuation of the ultrasonic echoes that varies depending on the positional relationship between the reception depth and the respective ultrasonic transducers within the aperture are corrected. Since the amount of correction differs depending on the part to be observed, standard values according to parts to be observed may be set as default values in advance, and the operator may change the setting value while watching the displayed image.

Furthermore, the signal preprocessing unit 122 may perform processing such as smoothing on the corrected reception signals.

The reception delay control unit 123 has plural delay patterns (phase matching patterns) corresponding to the reception direction and focal depth of the ultrasonic echoes, and selects delay patterns to be provided to the plural reception signals according to the reception direction and focal depth that have been set by the aperture diameter setting unit 113 and supplies them to the tissue-by-tissue phasing addition method determining unit 130, the tissue-by-tissue phasing addition processing unit 133, and the phasing addition processing unit 140. A group of reception signals representing ultrasonic echoes generated within the object are determined by the delay patterns supplied from the reception delay control unit 123. These groups of reception signals include ultrasonic information on the regions where the ultrasonic echoes have been generated.

The tissue-by-tissue phasing addition method determining unit 130 includes a reflection distribution calculating unit 131 and a reflection signal evaluating unit 132, and determines one or more kind of phasing addition method to be used for generating a B-mode image representing different tissues with respect to a group of reception signals relating to a certain region within the object. The operation of the tissue-by-tissue phasing addition method determining unit 130 will be described later in detail.

The tissue-by-tissue phasing addition processing unit 133 includes a variable amplifying unit 134 and a phasing addition unit 135, and performs phase matching on the group of reception signals and adds them to each other according to the tissue-by-tissue phasing addition method determined by the tissue-by-tissue phasing addition method determining unit 120. By the phasing addition processing (reception focus processing), at least one kind of sound ray data in which focal points of ultrasonic echoes are narrowed is formed. The sound ray data is accumulated in the first to N-th tissue-by-tissue B-mode image data generating units 136a, 136b, . . . according to the used tissue-by-tissue phasing addition method.

Each of the first to N-th tissue-by-tissue B-mode image data generating units 136a, 136b, . . . performs envelope detection processing on the waveform represented accumulated sound ray data and performing STC (sensitivity time gain control) processing thereon to generate image data representing values of pixels (brightness values) forming an ultrasonic image, and further performs DSC (digital scan converter) processing for converting the scan format of the image data. Thereby, the image data representing image information in the sound ray direction in the scan space of the ultrasonic beam is converted into image data for display in physical space. That is, in the DSC processing, resampling in correspondence with the image display range, and coordinate transformation and interpolation in correspondence with the scan format of ultrasonic waves. For example, on the image data obtained by linear scan, interpolation processing for generating linear images is performed. Further, on image data obtained by sector scan, convex scan, or radial scan, polar coordinate transformation and interpolation processing are performed.

By the processing of the first to N-th tissue-by-tissue B-mode image data generating units 136a, 136b, . . . , image data representing a B-mode image, in which different tissues are separated, such as a B-mode image representing surfaces of hard tissues such as bone parts, a B-mode image representing soft tissues such as muscle tissues and blood vessels, or a B-mode image representing speckle components is generated.

The image synthesizing unit 137 generates synthesized image data by superimposing plural kinds of tissue-by-tissue image data respectively generated in the first to N-th tissue-by-tissue B-mode image data generating units 136a, 136b, . . . . In this regard, addition ratios may be varied with respect to each tissue. Alternatively, the image synthesizing unit 137 may superimpose plural kinds of tissue-by-tissue image data, or handle selected one kind of tissue-by-tissue image data as synthesized image data without change, under the control of the control unit 110. The operator can select tissue-by-tissue image data to be superimposed and adjust brightness values (density) of the respective tissue-by-tissue images by using the tissue information enhancement input section 112a of the operation panel 112. Thereby, the operator can display only a desired tissue on a screen or emphasize a desired tissue in an ultrasonic image in which plural tissues are displayed.

The color signal generating unit 138 generates color signals for displaying the B-mode image in different colors by different tissues based on the plural kinds of tissue-by-tissue image data respectively generated in the first to N-th tissue-by-tissue B-mode image data generating units 136a, 136b, . . . . For example, blue color signals are generated based on B-mode image data representing hard tissues, red color signals are generated based on B-mode image data representing soft tissues, and yellow color signals are generated based on B-mode image data representing speckle components.

The phasing addition processing unit 140 matches phases of the plural reception signals that have been A/D converted and preprocessed according to need and adds them to each other based on the delay pattern supplied from the reception delay control unit 123. By the phasing addition processing, sound ray data in which focal points of ultrasonic echoes are narrowed is formed.

The B-mode image data generating unit 141 generates B-mode image data representing values of pixels forming an ultrasonic image by performing envelope detection processing and STC processing, and further generates B-mode image data for display by converting the scan format (DSC processing) of the B-mode image data.

The display image control unit 151 controls the display format for displaying on the screen a tissue-by-tissue synthesized image represented by the synthesized image data generated in the image synthesizing unit 137 and a normal B-mode image represented by the B-mode image data generated in the B-mode image data generating unit 141. As display formats, there are a format for selecting and displaying one of the tissue-by-tissue synthesized image and the normal B-mode image, a format for arranging and displaying two ultrasonic images side-by-side, etc. Further, the normal B-mode image may be displayed in different colors by tissue using the color signals generated in the color signal generating unit 138. These display formats may be automatically designated in advance, or manually set by the operator using the operation panel 112. Further, the display image control unit 151 may perform image processing such as gradation processing on the synthesized image data and B-mode image data.

The display unit 151 includes a display device such as a CRT or LCD, and displays ultrasonic images under the control of the display image control unit 151.

Next, a method of generating the tissue-by-tissue B-mode image data will be described.

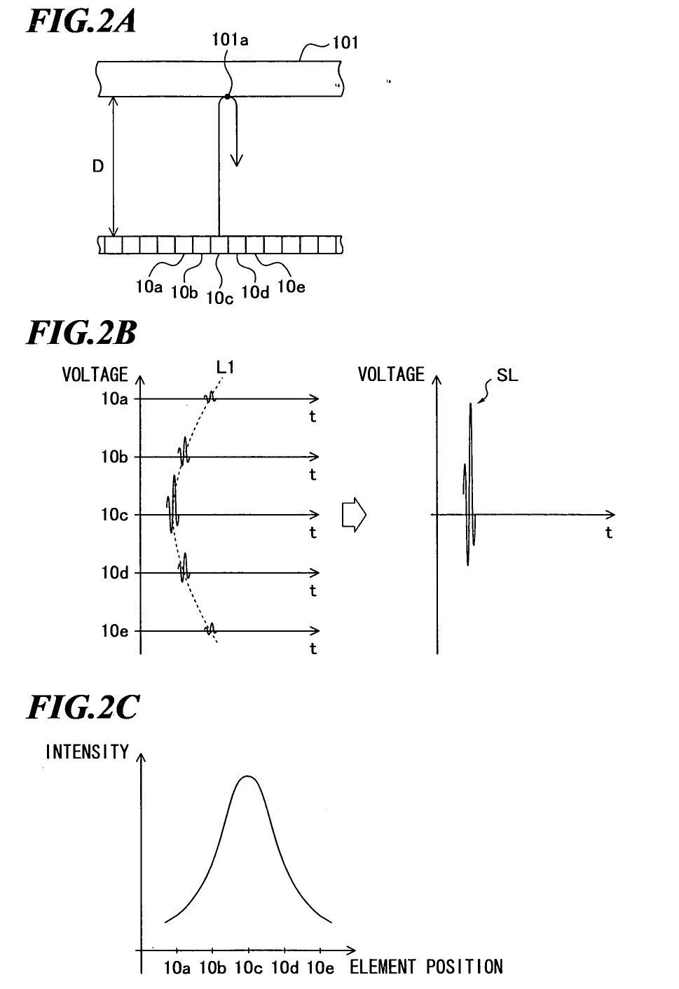

FIGS. 2A to 4D are diagrams for explanation of a principle of acquiring tissue information of the object.

As shown in FIG. 2A, the case will be considered where an ultrasonic beam is transmitted toward a reflector 11 and an ultrasonic echo reflected on the surface of the reflector 11 located at depth “D” is received by using an ultrasonic transducer array including ultrasonic transducers 10a to 10e. FIG. 2B shows reception waveforms of ultrasonic echoes at the ultrasonic transducers 10a to 10e. In FIG. 2B, the horizontal axis indicates time (t) and the vertical axis indicates voltage of the reception signals. Further, FIG. 2C shows an intensity distribution of the reception signals output from the ultrasonic transducers 10a to 10e. In FIG. 2C, the horizontal axis indicates positions of ultrasonic transducers (elements) and the vertical axis indicates intensity of the reception signals.

The ultrasonic echoes reflected at reflection point 11a are first received by the ultrasonic transducer 10c right opposite to the reflection point 11a, and then, sequentially received by the ultrasonic transducers 10b and 10d and the ultrasonic transducers 10a and 10e as shown in FIG. 2B. In the case where the B-mode image is generated, a predetermined delay times are provided to the reception signals on the same phase matching line L1 and added them. Thereby, sound ray signal SL representing ultrasonic information on the reflection point 111a is formed.

In the case where the reflector 11 is a hard tissue such as a bone part, the ultrasonic waves are mainly reflected on the surface thereof in the direction in which they have been transmitted with little scattering. Further, since the reflectance on the surface of the hard tissue is high, the intensity of ultrasonic echoes becomes relatively high. Accordingly, a relatively sharp peak appears in the position of the ultrasonic transducer 10c in the intensity distribution of the reception signals as shown in FIG. 2C. Hereinafter, such a reflector as the reflector 11 that reflects ultrasonic waves mainly in one direction with little scattering reflection is referred to as a “specular reflector”, and the degree that the reflection directions of ultrasonic waves are concentrated on one direction, i.e., the degree that the scattering reflection is low is referred to as a “specular reflectance”. Generally, a reflector having a high specular reflectance is a hard tissue.

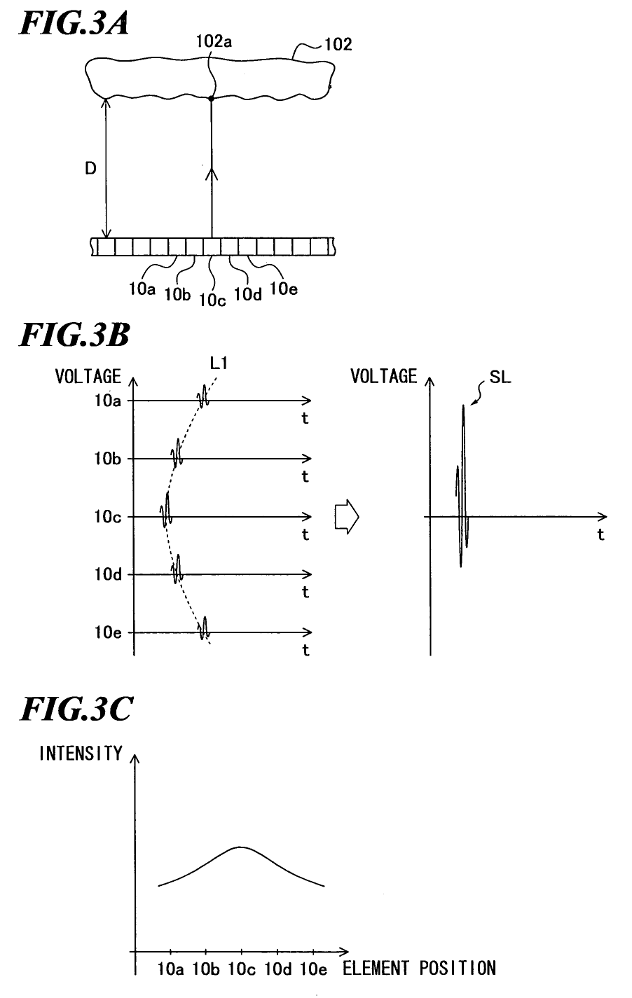

Next, as shown in FIG. 3A, the case will be considered where an ultrasonic beam is transmitted to a soft tissue such as a muscle or blood vessel. Generally, since a reflector of soft tissue readily reflects ultrasonic waves, and when an ultrasonic beam is transmitted toward a reflector 12 of soft tissue located at depth “D”, the ultrasonic beam is scattered in various directions at reflection point 12a. Thus generated ultrasonic echoes are received by the ultrasonic transducers 10a to 10e with timing depending on the depth “D” and the position of the reflection point 12a as shown in FIG. 3B. Since the timing is on the phase matching line L1 like the case of the reception waveform of the ultrasonic echoes shown in FIG. 2B, when phase matching is performed for generating a B-mode image, sound ray signal SL is formed like that shown in FIG. 2B.

However, since the intensity of ultrasonic echoes is dispersed in various directions due to scattering of ultrasonic waves in the soft tissue, the intensity distribution of the reception signals becomes relatively flat as shown in FIG. 3C. Hereinafter, such a reflector as the reflector 12 having a low specular reflectance (i.e., a high scattering reflection) is referred to as a “scattering reflector”.

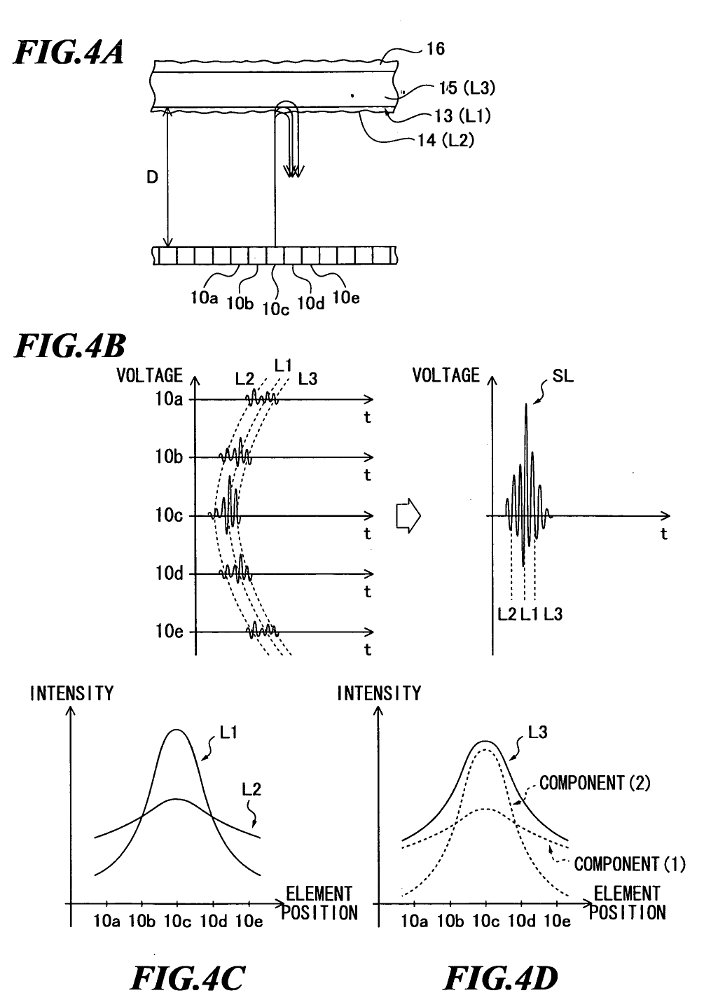

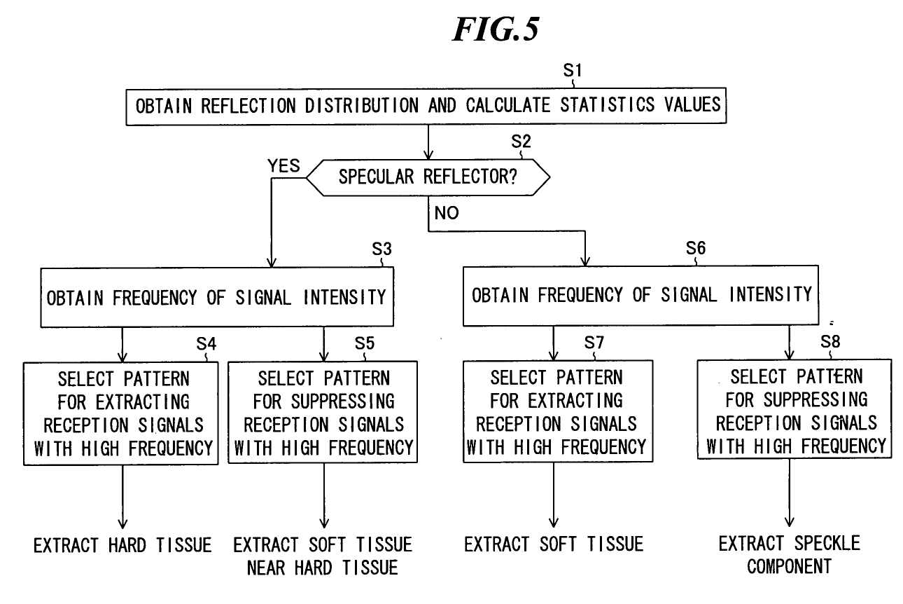

Next, the case of imaging a soft tissue existing near a hard tissue or a tissue behind a hard tissue will be considered. Specifically, as shown in FIG. 4A, the case corresponds to imaging of a region where a soft tissue 14 such as a muscle exists around a hard tissue surface 13 such as a bone, and a bone internal tissue 15 as a region of bone marrow, spongy bone structure, etc. exhibiting scattering reflection near that of a soft tissue. By transmitting ultrasonic waves to such regions, ultrasonic echoes are generated in the respective tissues.

As shown in FIG. 4B, a sound ray signal SL is obtained by performing phase matching on a group of reception signals on the uniform phase matching lines L1 to L3. In FIG. 4B, the reception signal on the phase matching line L1 represents an ultrasonic echo signal generated in the hard tissue 13, the reception signal on the phase matching line L2 represents an ultrasonic echo signal generated in the soft tissue 14, and the reception signal on the phase matching line L3 represents an ultrasonic echo signal generated in the bone internal tissue 15.

Here, since the reflectance in the hard tissue surface 13 is much larger than that of the surface of the soft tissue 14, in the case where the soft tissue 14 exists at the front side of the hard tissue surface 13, the ultrasonic echoes from the soft tissue 14 have relatively low impact on the ultrasonic echoes from the hard tissue surface 13. However, since the intensity of the reception signal on the phase matching line L1 becomes much larger than the reception signal on the phase matching line L2, when image signals obtained by performing phasing addition on those reception signals without change are displayed on the same display screen, the brightness of an image relating to the phase matching line L2 (i.e., an image representing the soft tissue 14) becomes relatively and significantly low, and it becomes difficult to visually recognize and discriminate the image from an image relating to the phase matching line L1 (i.e., the hard tissue surface 13).

Further, as shown in FIG. 4C, intensity distributions of the reception signals on the uniform phase matching lines L1 and L2 differ from each other. For example, the reception signals outputted from the ultrasonic transducers 10a to 10e located in a diagonal direction relative to the reflection point contains not so much signal components from the specular reflector. That is, in such reception signals, the intensity difference between the ultrasonic echo signal from the soft tissue 14 and the ultrasonic echo signal from the hard tissue surface 13 becomes small. Accordingly, by focusing attention on the ultrasonic transducers other than those near the central part containing signal components from the specular reflector, the soft tissue 14 near the hard tissue surface can be easily viewable in the ultrasonic image.

On the other hand, regarding the bone internal tissue 15, the influence (e.g., ringing or the like) by the ultrasonic echoes having large amplitude generated in the hard tissue surface 13 becomes problematic. That is, since the ultrasonic echoes reflected from the bone internal tissue 15 exhibiting scattering reflection near that of the soft tissue like bone marrow, spongy bone structure or the like originally have small amplitude, and the large-amplitude ultrasonic echoes affect more easily as the tissue is closer to the bone surface, the ultrasonic echoes from the internal tissue 15 are substantially buried. Accordingly, it is extremely difficult to image tissues existing at the rear side of a soft tissue by the method of generating normal B-mode image.

As shown in FIG. 4D, the intensity distribution of the group of reception signals on the phase matching line L3 shows an approximate distribution to that of the specular reflector as a whole. However, each reception signal includes a component (1) of an ultrasonic echo signal from the internal tissue 15 and a component (2) due to influence of the ultrasonic echo signal (large-amplitude signal) from the hard tissue surface 13. Among them, the intensity distribution of the component (1) exhibits a feature as a scattering reflector like a soft tissue surface and the intensity distribution of the component (2) exhibits a feature as a specular reflector like a hard tissue surface, and thereby, the intensity distributions of both components are different.

Accordingly, by focusing attention on the component ratio in each reception signal, for example, the reception signal received by the ultrasonic transducer 10c nearly right opposite to the reflection point of the ultrasonic wave includes many components (2) due to influence of the large-amplitude signals. Contrary, the reception signals received by the ultrasonic transducer 10a or 10e located in the diagonal direction relative to the reflection point includes less components (2) due to influence of the large-amplitude signals and more scattering components (1) from the internal tissue 15. Thus, by focusing attention to the difference of components between reception signals, ultrasonic echoes representing the regions at the rear side of the hard tissue that have been buried due to large-amplitude signals can be extracted.

Similarly, also ultrasonic echoes from a soft tissue 16 (FIG. 4A) existing at the rear side of the hard tissue such as a bone part can be extracted.

As shown in FIGS. 2A to 4D, by focusing attention on the mutual property (interrelationship) of a group of reception signals relating to a certain region, unlike the case where a B-mode image is generated simply by phase matching the reception signals, the tissue property of the region can be determined and a region with small reflectance (soft tissue) existing near a region with large reflectance (hard tissue) can be extracted.

FIG. 5 is a diagram for explanation of the operation of the tissue-by-tissue phasing addition method determining unit 130 shown in FIG. 1.

First, at step S1, the reflection distribution calculating unit 131 of the tissue-by-tissue phasing addition method determining unit 130 obtains a spatial intensity distribution of a group of reception signals on the same phase matching line of the plural reception signals processed in the signal preprocessing unit 122. That is, in a graph with the horizontal axis as position coordinate of transducer and the vertical axis as intensity of reception signal, intensity of the reception signals on the same phase matching line output from the plural ultrasonic transducers within aperture diameter DA of the ultrasonic transducers is plotted. The group of reception signals on the same phase matching line are determined based on the delay pattern supplied from the reception delay control unit 123. Further, hereinafter, the reflection points where ultrasonic echoes (reflection signals) represented by these reception signals are generated is referred to as an analysis region, and the spatial intensity distribution of a group of reception signals on the same phase matching line is referred to as a reflection distribution.

Further, the reflection distribution calculating unit 131 calculates predetermined statistics values based on the obtained reflection distribution. In this regard, in the previously obtained reflection distribution, the horizontal axis is read as data value and the vertical axis is read as frequency from a different perspective. Thus obtained relationship diagram is handled as a frequency distribution chart representing the relationship between random probability x and probability density function f(x).

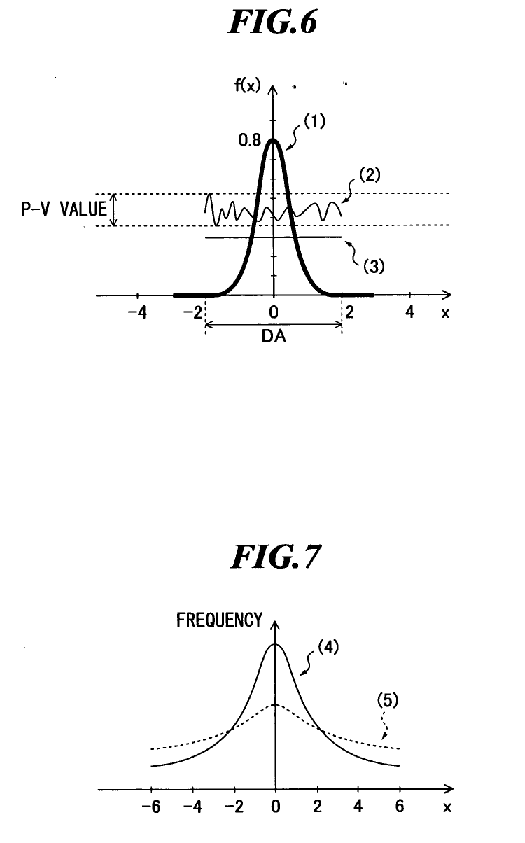

As shown in FIG. 6, curve (1) represents a frequency in the case where the frequency distribution is concentrated on a certain value, that is, a frequency distribution of a group of reception signals representing ultrasonic echoes reflected by a specular reflector. Further, curve (2) represents a frequency distribution in the case where the frequency is randomly distributed, that is, a frequency distribution of a group of reception signals representing ultrasonic echoes reflected by a scattering reflector. Furthermore, curve (3) shown for comparison represents a frequency distribution in the virtual case where ultrasonic echoes propagate from plural directions with equal intensity.

For example, the statistics values calculated in the reflection signal evaluating unit 132 are as follows:

(1) Mean

A mean is used as a value representing quantitative characteristics of frequency. When an ultrasonic echo propagating from the front direction of the ultrasonic transducer array is received, the mean typically becomes zero (center), while, when a reflector is inclined relative to the ultrasonic transducer array, the mean is shifted from the center toward an end. Not only the typical arithmetic mean but also median or mode is used as mean. Since the magnitude relationship between these arithmetic means, medians, or modes changes according to the distribution conditions of frequency, they can be used when variations in frequency are estimated.

(1-1) Median

A median refers to a value located at the center of the number of data in the case where the frequencies are arranged in order from the minimum value. When the number of data is even, the arithmetic mean of the center two values is used.

(1-2) Mode

A mode refers to a value with the highest frequency among frequencies.

(2) Variance

A variance is one of scales that indicate variations in frequency, and obtained by dividing sum of squares of deviation as differences between the respective detection data and arithmetic mean by the number of data (or the number of data −1). When the frequency distribution is close to the normal distribution and the peak rises as the curve (1), a variance value becomes smaller. Contrary, when the frequency distribution is random as the curve (2) or when the frequency distribution is uniform as the curve (3), a variance value becomes larger.

(3) Skewness

A skewness refers to a scale that indicates the degree of asymmetry around the mean of frequency, and is obtained by the following expression.
Skewness=(sum of cube of deviation)/(number of data)/(cube of standard deviation)

Zero of skewness represents that the frequency distribution is not deviated, and, in this case, the arithmetic mean, the median, and the mode become equal. Further, positive skewness represents that the frequency distribution is negatively deviated, and, in this case, the relationship arithmetic mean>median>mode holds. Furthermore, negative skewness represents that the frequency distribution is positively deviated, and, in this case, the relationship arithmetic mean<median<mode holds.

(4) Kurtosis

A kurtosis refers to a scale that indicates degree of concentration around the mean of frequency (sharpness), and is obtained by the following expression.
Kurtosis=(sum of biquadrate of deviation)/(number of data)/(biquadrate of standard deviation)

Here, in a standard normal distribution having a mean of “0” and variance of “1”, the kurtosis becomes “3”. Accordingly, the kurtosis is evaluated with a numeric value “3” as reference. That is, when the kurtosis is “3”, the frequency distribution is close to the normal distribution. Further, the smaller than “3” the kurtosis becomes, flatter the frequency distribution becomes. Furthermore, the larger than “3” the kurtosis becomes, sharper the frequency distribution around the mean becomes.

(5) P-v Value, Square Mean Between Adjacent Elements, Etc.

When the frequency is randomly distributed as the curve (2), a scale indicating the degree of random is also calculated. As such a scale, for example, as shown in FIG. 6, a distance between a peak and a valley (p-v value) in the curve (2), difference square mean between adjacent ultrasonic transducers or the like is used. These scales show that, the larger the value, the more indefinite the ultrasonic echo is and larger the speckle component is.

The reference values (threshold values or the like) for determining the features of the reflection distribution based on the statistics values of these (1) to (5) are stored in the tissue-by-tissue reflection information storage section 111a.

Referring to FIG. 5 again, at step S2, the reflection signal evaluating unit 132 determines tissue property of the analysis region based on the statistics values calculated at step S1. When the determination is made, tissue information stored in the tissue-by-tissue reflection information storage section 111a is referred to. For example, as shown in curve (4) in FIG. 7, in the case where the variance of the reflection distribution is smaller than a threshold value or the kurtosis is larger than a threshold value, the analysis region is determined as a specular reflector. Contrary, as shown in curve (5) in FIG. 7, in the case where the variance of the reflection distribution is larger than the threshold value, the analysis region is determined as a scattering reflector (that is, not a specular reflector).

Alternatively, not the determination whether or not the region is a specular reflector is performed by comparing the statistics values calculated at step S1 with the reference values, but degree of specular reflection components in the analysis region (specular reflectance in the analysis region) may be obtained based on the statistics values.

At step S2, in the case where an analysis region is determined as a specular reflector, or the specular reflectance of an analysis is high, the reflection signal evaluating unit 132 obtains the frequency of signal intensity in the reflection intensity at step S3.

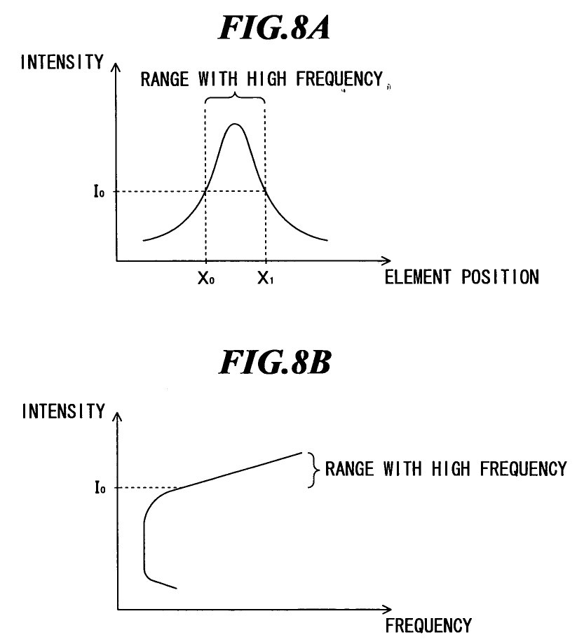

FIG. 8A shows a reflection distribution in an analysis region determined as a specular reflector, and FIG. 8B shows a frequency of signal intensity created based on the reflection distribution. As shown in FIG. 8B, it is considered that a range where the frequency of signal intensity is relatively high (e.g., a range where the signal intensity is equal to or more than I₀) represents the feature of the analysis region. Accordingly, the feature of the analysis region can be extracted, or contrary, the feature can be suppressed to raise other elements by controlling the handling of the signals contained in the range with high frequency. Specifically, the scattering components from the soft tissue, much of them are contained at the ends of the reflection distribution (see FIGS. 4A to 4D), can be clarified by suppressing the reception signals in the range with high frequency of signal intensity.

At step S4, the reflection signal evaluating unit 132 controls the tissue-by-tissue phasing addition processing unit 133 to perform phasing addition of reception signals contained in a range with relatively low frequency of signal intensity, i.e., reception signals outputted from the elements located in a range except X₀ to X₁ shown in FIG. 8A with lowered gain. Thereby, the reception signals in the range with high frequency of signal intensity, i.e., reception signals mainly contain ultrasonic echoes from the hard tissue are extracted.

Further, at step S5, the reflection signal evaluating unit 132 controls the tissue-by-tissue phasing addition processing unit 133 to perform phasing addition of reception signals contained in a range with relatively high frequency of signal intensity, i.e., reception signals outputted from the elements located in a range of X₀ to X₁ (near the center of the reflection distribution) shown in FIG. 8A with lowered gain. Thus, by suppressing the reception signals in the range with high frequency of signal intensity, reception signals contained in the range with low frequency (both ends of the reflection intensity) are relatively raised. Thereby, reception signals mainly contain ultrasonic echoes from the soft tissue are extracted.

On the other hand, at step S2, in the case where an analysis region is determined as a scattering reflector, or the specular reflectance of an analysis is low, the reflection signal evaluating unit 132 obtains the frequency of signal intensity in the reflection intensity at step S6.

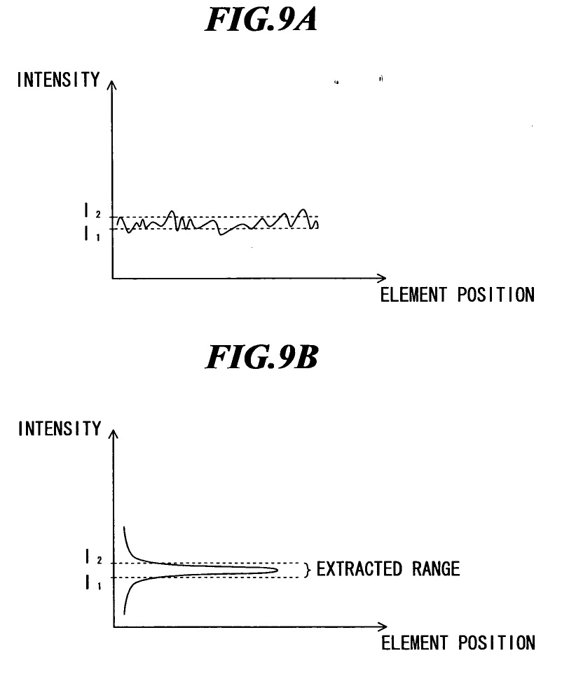

FIG. 9A shows a reflection distribution in the scattering reflector with relatively small variations in reception signals, and FIG. 9B shows a frequency of signal intensity created based on the reflection distribution. As shown in FIG. 9B, in the case where variations in reception signals are relatively small, a relatively sharp peak appears. It is considered that the analysis region represented by such group of reflection signals is a relatively uniform tissue, and the tissue is generally a substantial soft tissue such as a flesh and blood vessel.

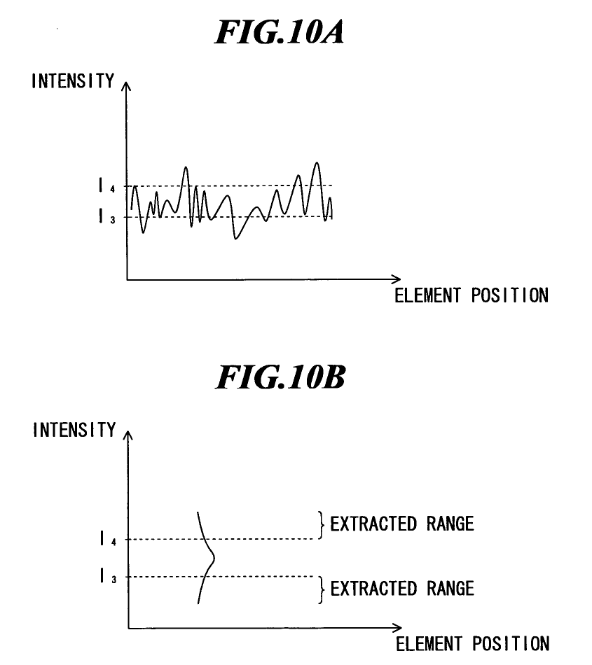

On the other hand, FIG. 10A shows a reflection distribution in the scattering reflector with relatively large variations in reception signals, and FIG. 10B shows a frequency of signal intensity created based on the reflection distribution. As shown in FIG. 10B, in the case where variations in reception signals are relatively large, a gentle peak appears. It is considered that the analysis region represented by such group of reflection signals is not a substantial soft tissue, and the tissue is a speckle containing many unstable signals.

Accordingly, in the case where an analysis region is a scattering reflector, the reception signals are extracted or suppressed according to the frequency of signal intensity, and thereby, a substantial soft tissue and a speckle component can be imaged separately.

At step S7, the reflection signal evaluating unit 132 controls the tissue-by-tissue phasing addition processing unit 133 to perform phasing addition of reception signals contained in a range with relatively high frequency of signal intensity, i.e., reception signals with signal intensity less than I₁ or more than I₂ as shown in FIG. 9B with lowered gain. Thereby, the reception signals formed by signal components that are relatively stable with the signal intensity within the range I₁ to I₂, i.e., reception signals mainly contain ultrasonic echoes from the soft tissue are extracted.

Further, at step S8, the reflection signal evaluating unit 132 controls the tissue-by-tissue phasing addition processing unit 133 to perform phasing addition of reception signals with signal intensity within the range I₃ to I₄ as shown in FIG. 10A with lowered gain. Thereby, reception signals with low frequency, that is, containing stable signal components (speckle components) are relatively raised.

As specific processing at each of these steps S4, S5, S7, and S8, the reflection signal evaluating unit 132 selects at least one appropriate amplification factor control pattern from among the plural amplification factor control patterns that have been stored in the signal amplification factor control pattern storage section 111b in advance, and supplies them to the variable amplifying unit 134 (FIG. 1). At step S2, in the case where the specular reflectance of the analysis region is middle (that is, the determination whether or not it is a specular reflector is hard), the reflection signal evaluating unit 132 may perform both processing at steps S3 and S6.

The variable amplifying unit 134 shown in FIG. 1 amplifies the group of reception signals based on the amplification factor control pattern supplied from the reflection signal evaluating unit 132 with gain determined with respect to respective reception signals. Thereby, one or plural groups of amplified reception signals are formed according to the type of amplification factor control pattern. The phasing addition unit 135 matches phases of the amplified reception signals in the respective groups by providing predetermined delays and adds them. Thereby, one or plural kinds of sound ray data are generated. Thus generated sound ray data are stored in one of the first to N-th tissue-by-tissue B-mode image data generating units 136a, 136b, . . . according to the type of amplification factor control pattern.

FIG. 11 is a schematic diagram showing an ultrasonic image generated by the ultrasonic imaging apparatus according to the embodiment. As shown in FIG. 11, in the ultrasonic image, especially, a soft tissue 112 existing near a hard tissue such as a bone part 111 can be clearly imaged. Further, a substantial soft tissue 113 such as a muscle and blood vessel and an insubstantial speckle region 114 can be imaged separately from each other. Furthermore, the ultrasonic image can be made more easily viewable by displaying a B-mode image in different colors according to the kinds of tissue property.

As described above, according to the first embodiment of the present invention, by varying the gain of a group of reception signals with respect to respective reception signals according to a reflection distribution of the group of reception signals representing ultrasonic echoes generated at a certain reflection point, reception signals mainly containing signal components relating to a desired tissue can be extracted. Thereby, the desirable tissue can be appropriately displayed as an ultrasonic image. Therefore, in the case where a structure largely different in intensity of ultrasonic echoes exists nearby, a structure of target tissue can be imaged. Further, by selectively suppressing the signal components from a region with very large reflectance like a bone part, an ultrasonic image easily viewable as a whole can be generated.

Further, according to the embodiment, plural tissue-by-tissue B-mode images can be generated by varying the amplification factor control pattern to be applied to the group of reception signals. Thereby, an ultrasonic image in which only desired tissues are combined, an ultrasonic image in which a desired tissue is emphasized, an ultrasonic image in which different tissues are displayed in different colors, etc. can be displayed according to the purpose of medical diagnoses and the preference of users. Furthermore, since such tissue-by-tissue B-mode images or a synthesized image thereof and a normal B-mode image can be displayed simultaneously, or while selectively switching one of them, the diagnostic efficiency can be improved.

In addition, according to the embodiment, tissue property of a reflector that has generated ultrasonic echoes can be evaluated by simple calculation by utilizing a spatial intensity distribution (reflection distribution) of a group of reception signals and statistics values thereof. Therefore, the tissue-by-tissue B-mode images can be displayed in real time.

By the way, in the first embodiment of the present invention, the analysis of reflection distribution of reception signals has been further performed (steps S3 and S6) after the determination as to whether or not the reflector is a specular reflector at step 2 shown in FIG. 5, however, those two stages of processing may be simultaneously performed. In this case, plural kinds of signal amplification factor control patterns may be stored in association with mutual property (reflection information) of the group of reception signals.

Next, an ultrasonic imaging apparatus according to the second embodiment of the present invention will be described. In the ultrasonic imaging apparatus according to the embodiment, the processing in the tissue-by-tissue phasing addition method determining unit 130 shown in FIG. 1 is different from that of the ultrasonic imaging apparatus according to the first embodiment. That is, the embodiment is characterized by analyzing a reflection distribution of reception signals based on the shape of a histogram corresponding to the reflection distribution. Other constitution is the same as that in the first embodiment of the present invention.

The reflection distribution calculating unit 131 shown in FIG. 1 creates a reflection distribution based on a group of reception signals on the same matching line, which have been subjected to predetermined processing in the signal preprocessing unit 122, and creates a histogram based on the reflection distribution.

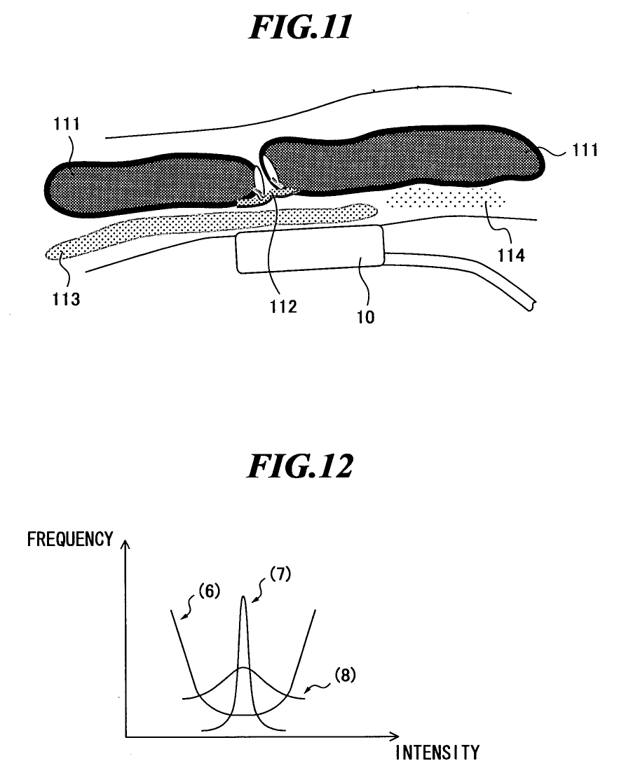

Here, as shown by curves (6) to (8) in FIG. 12, histogram shapes corresponding to reflection distributions are generally classified into three shapes.

Curve (6) is a histogram corresponding to a specular reflector as shown in FIG. 8A. In this case, since the reception signals are concentrated in a range with high intensity and/or a range with low intensity, the shape of the histogram becomes U-shaped. The analysis region showing such a reflection distribution is generally a hard tissue, and the same reflection distribution is shown in the case where a soft tissue exists near a hard tissue.

Curve (7) is a histogram corresponding to a scattering reflector with relatively small variations as shown in FIG. 9A. In this case, since the intensity of reception signals is concentrated in a narrow range to some degree, the shape of the histogram becomes single-peaked with a sharp peak. The analysis region showing such a reflection distribution is generally a soft tissue.

Curve (8) is a histogram corresponding to a scattering reflector with relatively large variations as shown in FIG. 10A. In this case, since the intensity of reception signals is concentrated in a broad range to some degree, the shape of the histogram becomes single-peaked with a relatively gentle peak. In the case where such a reflection distribution is shown, a speckle pattern generally appears.

The reflection signal evaluating unit 132 shown in FIG. 1 judges whether or not an analysis region is a specular reflector by determining the shape of a histogram using pattern matching, similarity determination using the least square method or the like, similarity determination to theoretical values of statistical parameters, etc., and selects an amplification factor control pattern to be applied to a group of reception signals. In this case, mode, median, r-th moment about mean can be used as the statistical parameters.

The amplification factor control pattern to be applied to a group of reception signals is the same as that has been described in the first embodiment by referring to FIGS. 8A to 10B. Further, those amplification factor control patterns have been stored in the amplification factor control pattern storage section 111b in association with histogram shapes.

As a modified example of the ultrasonic imaging apparatus according to the second embodiment of the present invention, various statistics values may be calculated based on the histogram corresponding to a reflection distribution of reception signals, and select an amplification factor control pattern to be applied to a group of reception signals. As the statistics values, mode, median, quartile deviation, skewness, frequency, etc. are used. Here, the quartile deviation is an indicator representing the degree of scattering of frequency, and the quartile deviation QR is obtained by the following expression using the first quartile X_{0.25} and the third quartile X_{0.75}. The quartile is a value in a position where the frequency is divided into quarters when data is aligned in ascending order, and the first quartile is a value located at 25% in ascending order and the third quartile is a value located at 75% in ascending order.
QR=(X_{0.75}−X_{0.25})/2

Next, an ultrasonic imaging apparatus according to the third embodiment of the present invention will be described. In the ultrasonic imaging apparatus according to the embodiment, the processing in the tissue-by-tissue phasing addition method determining unit 130 shown in FIG. 1 is different from those in the ultrasonic imaging apparatuses according to the first and second embodiments. That is, the embodiment is characterized by analyzing a histogram corresponding to a reflection distribution using beta distribution. Other constitution is the same as that in the first embodiment of the present invention.

The reflection distribution calculating unit 131 shown in FIG. 1 creates a reflection distribution based on a group of reception signals on the same matching line, which have been subjected to predetermined processing in the signal preprocessing unit 122, and creates a histogram based on the reflection distribution (see FIG. 12). Further, the unit normalizes the created histogram so that the range of values (the horizontal axis of the histogram) may be “0” to “1”.

Then, the reflection distribution calculating unit 131 qualifies the distribution condition of the normalized histogram using beta distribution. Here, the beta distribution is expressed using shape parameters α and β by X˜B(α, β) and probability density function f(x) in the beta distribution, rth moment (product moment) about origin, mean E(x), variance VAR(x), and mode MOD are expressed by the following expressions (1) to (5).

$$\begin{matrix}f ( x ) = \frac{1}{B ( α , β )} {{x}^{α - 1} ( 1 - x )}^{β - 1} ( 0 \leq x \leq 1 ) & ( 1 ) \\ {μ}_{r} = \frac{B ( α + r , β )}{B ( α , β )} ( r \geq 1 ) & ( 2 ) \\ E ( x ) = \frac{α}{α + β} & ( 3 ) \\ VAR ( x ) \frac{α β}{{( α + β )}^{2} ( α + β + 1 )} & ( 4 ) \\ MOD = \frac{α - 1}{α + β - 2} ( α > 1 , β > 1 ) & ( 5 )\end{matrix}$$

In order to obtain the beta distribution, sample mean x_{AVE} and variance σ² are obtained using the following expressions (6) and (7) from the normalized histogram.

$$\begin{matrix}{x}_{AVE} = \frac{1}{N} {\sum }_{i = 1}^{n} {f}_{i} {m}_{i} & ( 6 ) \\ {σ}^{2} = \frac{1}{N} {\sum }_{i = 1}^{n} {f}_{i} {m}_{i}^{2} - {x}_{AVE}^{2} & ( 7 )\end{matrix}$$

Then, the reflection distribution calculating unit 131 obtains beta distribution parameters α and β by estimation according to the moment method using the following expressions (8) and (9)

$$\begin{matrix}α \text{:} {x}_{AVE} \{ [ {x}_{AVE} ( 1 - {x}_{AVE} ) / ( \frac{n - 1}{n} ) {σ}^{2} ] - 1 \} & ( 8 ) \\ β \text{:} ( 1 - {x}_{AVE} ) \{ [ {x}_{AVE} ( 1 - {x}_{AVE} ) / ( \frac{n - 1}{n} ) {σ}^{2} ] - 1 \} & ( 9 )\end{matrix}$$

Thereby, an approximate distribution to the beta distribution is obtained.

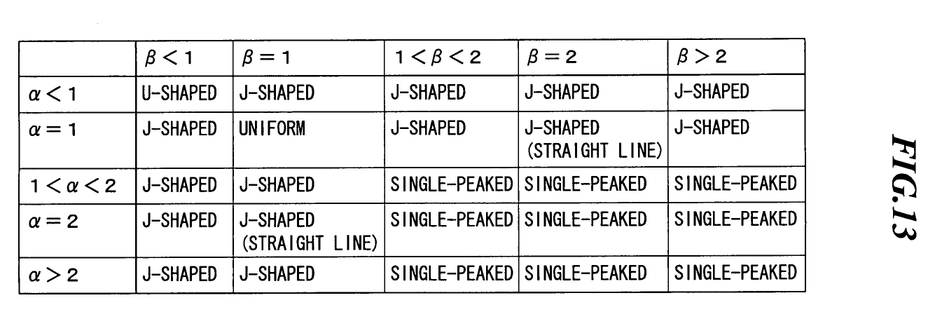

Then, the reflection signal evaluating unit 132 selects an amplification factor control pattern to be applied to the group of reception signals corresponding to the analysis region according to the values of α and β. FIG. 13 is a chart showing classified parameters of beta distribution. “U-shaped”, “J-shaped”, and “single-peaked” in FIG. 13 represent shapes of the probability density function in the beta distribution.

(i) The Case where α<1 and β<1

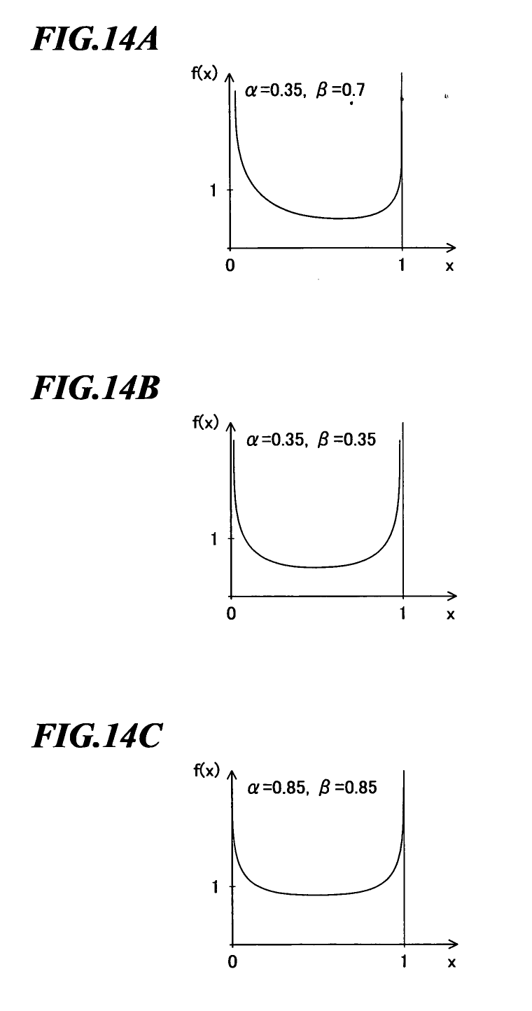

In this case, as shown in FIGS. 14A to 14C, the probability density function f(x) becomes U-shaped. The peak rises in the intensity distribution of reception signals (see FIG. 8A) and this represents that the reflector surface is a specular reflector. Accordingly, the reflection signal evaluating unit 132 selects an amplification factor control pattern for extracting reception signals with high frequency for imaging a hard tissue, and selects an amplification factor control pattern for suppressing reception signals with high frequency for imaging a soft tissue existing near the hard tissue.

Here, the intensity of specular reflection in the analysis region changes according to the value |α×β|. For example, as shown in FIG. 14A or 14B, the smaller the value |α×β| is, the steeper the U-shaped gradient of the probability density function f(x) becomes. Contrary, as shown in FIG. 14C, the larger the value |α×β| is, the gentler the U-shaped gradient of the probability density function f(x) becomes and the weaker the specular reflection becomes. Accordingly, the reflection signal evaluating unit 132 selects the amplification factor control patterns that differ in ranges of reception signals with gain to be adjusted and adjustment amounts according to the value |α×β|.

(ii) The Case where (α−1)×(β−1)≦0

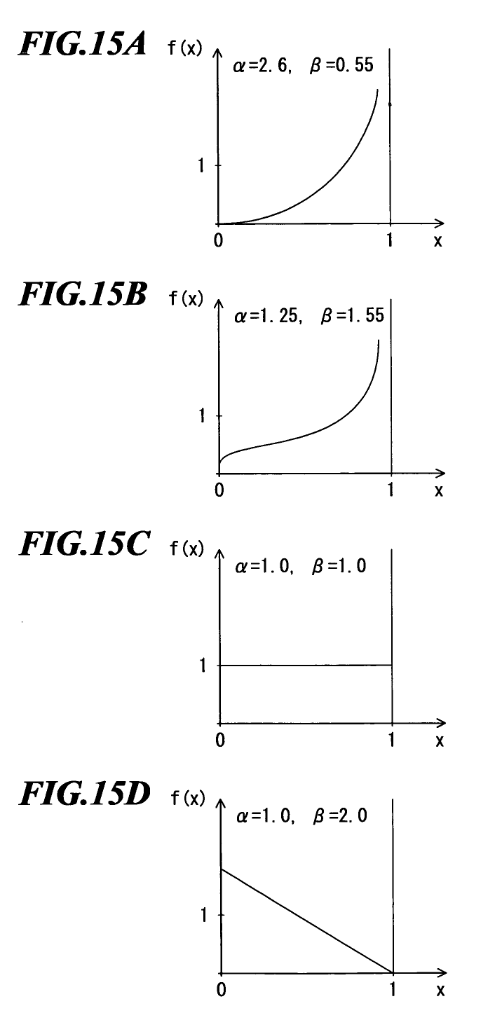

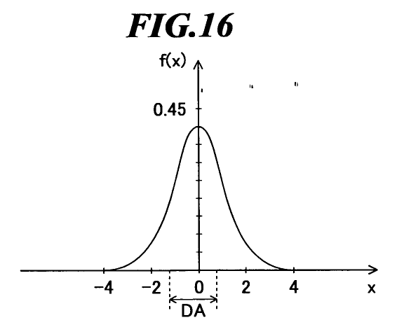

In this case, as shown in FIGS. 15A to 15D, the probability density function becomes J-shaped. This represents that the specular reflection has a peak rising to some degree in the intensity distribution of reception signals (i.e., a specular reflector) and the peak center of intensity (x=0) resides outside of the aperture DA of the transducer array as shown in FIG. 16. Such a reflection distribution is seen in the case where ultrasonic echoes propagating from the diagonal direction relative to the ultrasonic transducer array. Accordingly, also in this case, a hard tissue and/or a soft tissue existing near the hard tissue can be imaged by selecting appropriate amplification factor control patterns.

Further, in this case, the intensity of specular reflection in the analysis region changes according to the value |α/β|. For example, as shown in FIG. 15A or 15B, the more distant from “1” the value |α/β| is, the steeper the gradient of the J-shape becomes. Contrary, as shown in FIG. 15C or 15D, the closer to “1” the value |α/β| is, the gentler the gradient of the J-shape becomes (e.g., gradient “0”) and represents weaker specular reflection. Accordingly, the reflection signal evaluating unit 132 selects the amplification factor control patterns that differ in ranges of reception signals with gain to be adjusted and adjustment amounts according to the value |α/β|.

(iii) The Case where α>1 and β>1

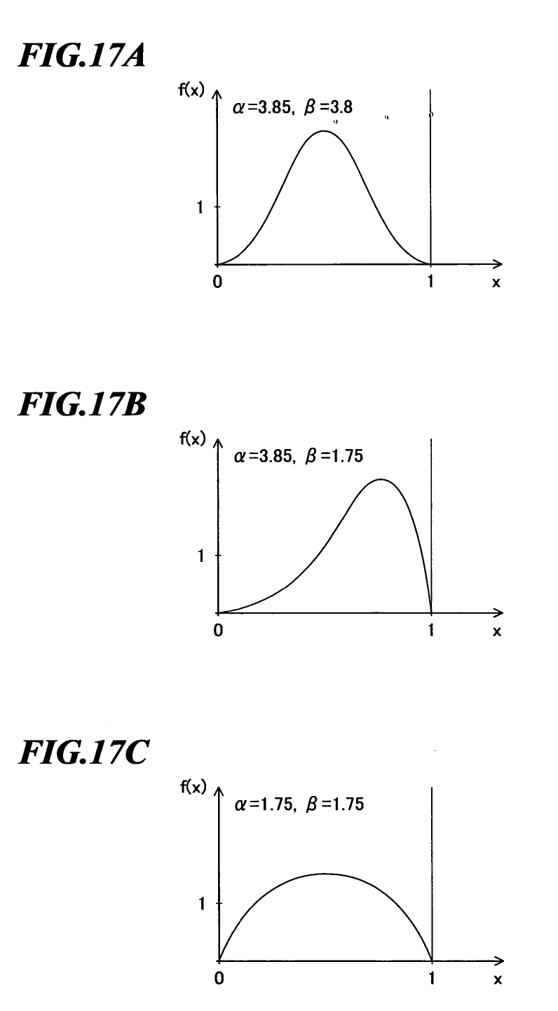

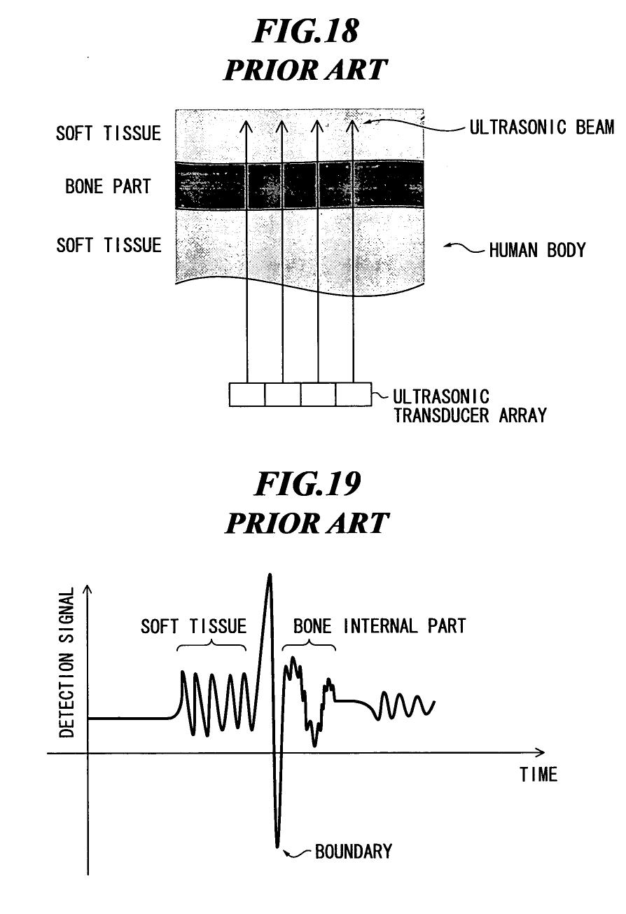

In this case, as shown in FIGS. 17A to 17C, the probability density function f(x) becomes single-peaked. This represents that the frequency of reception signals is a normal distribution (see FIGS. 9B and 10B) and the analysis region is a scattering reflector. Accordingly, the reflection signal evaluating unit 132 selects an amplification factor control pattern for extracting reception signals with high frequency for imaging a soft tissue, and selects an amplification factor control pattern for suppressing reception signals with high frequency for imaging speckle components.

Further, in this case, the larger the value |α/β| is, the steeper the peak of the probability density function f (x) becomes, that represents a small diffusion surface with small variations in intensity distribution. Contrary, the smaller the value |α/β| is, the gentler the peak of the probability density function f(x) becomes, and variations in intensity distribution become larger. Accordingly, the reflection signal evaluating unit 132 selects the amplification factor control patterns that differ in ranges of reception signals with gain to be adjusted and adjustment amounts according to the value |α/β|.

As described above, according to the third embodiment of the present invention, the reflection distribution can be analyzed correctly with simple calculation by utilizing the beta distribution obtained based on the histogram corresponding to the reflection distribution of reception signals. Therefore, tissue-by-tissue B-mode images can be generated in real time.

In the third embodiment of the present invention, the amplification factor control pattern to be applied to the group of reception signals has been selected by analyzing the histogram using beta distribution, however, the amplification factor control pattern may be directly selected based on the parameters α and β of beta distribution.

The calculation processing means for performing calculation and evaluation of the reflection distribution that has been described in the above first to third embodiments can be added to a general ultrasonic imaging apparatus as an advanced feature. Therefore, a system for generating tissue-by-tissue B-mode images can be formed at low cost.

1. An ultrasonic imaging apparatus comprising:
an ultrasonic probe including plural ultrasonic transducers for transmitting ultrasonic waves toward an object to be inspected and receiving ultrasonic echoes propagating from the object to output reception signals;
evaluating means for evaluating mutual property of a group of reception signals relating to a region within the object from among the reception signals respectively outputted from said plural ultrasonic transducers; and
variable amplifying means for amplifying said group of reception signals with signal amplification factors determined with respect to respective reception signals based on an evaluation result of said evaluating means.

2. An ultrasonic imaging apparatus according to claim 1, wherein said evaluating means obtains tissue property in a region, where the ultrasonic echoes propagating from the object are generated, based on said mutual property, and determines signal amplification factors of said group of reception signals with respect to respective reception signals according to the tissue property of said region.

3. An ultrasonic imaging apparatus according to claim 2, further comprising:
storage means for storing plural kinds of tissue information representing tissue property within the object and respectively associated with the mutual property of said group of reception signals representing the ultrasonic echoes,
wherein said evaluating means discriminates tissue property in the region, where the ultrasonic echoes propagating from the object are generated, based on said plural kinds of tissue information.

4. An ultrasonic imaging apparatus according to claim 1, further comprising:
second storage means for storing a plurality of signal amplification factor control patterns to be used when said group of reception signals are amplified with different signal amplification factors with respect to respective reception signals and associated with one of said mutual property and said plural kinds of tissue information,
wherein said evaluating means selects at least one signal amplification factor control pattern from among said plurality of signal amplification factor control patterns based on one of said mutual property and said plural kinds of tissue information; and
said variable amplifying means amplifies said group of reception signals according to said at least one signal amplification factor control pattern selected by said evaluating means.

5. An ultrasonic imaging apparatus according to claim 1, wherein:
said variable amplifying means amplifies said group of reception signals with at least one signal amplification factor control pattern to generate at least one group of amplified reception signals; and
said apparatus further comprises image data generating means for performing processing of matching phases of the reception signals and adding the reception signals to each other included in said at least one group of amplified reception signals generated by said variable amplifying means so as to generate at least one kind of B-mode image data.

6. An ultrasonic imaging apparatus according to claim 5, further comprising:
synthesized image data generating means for generating, when plural kinds of B-mode image data generated by said image data generating means are supplied, synthesized image data representing plural kinds of ultrasonic image information based thereon.

7. An ultrasonic imaging apparatus according to claim 5, further comprising:
second image data generating means for performing phasing addition processing on said group of reception signals to generate B-mode image data.

8. An ultrasonic imaging apparatus according to claim 6, further comprising:
second image data generating means for performing phasing addition processing on said group of reception signals to generate B-mode image data; and
display control means for selectively displaying on a display unit at least one of an ultrasonic image represented by the synthesized image data generated by said synthesized image data generating means and an ultrasonic image represented by the B-mode image data generated by said second image data generating means.

9. An ultrasonic imaging apparatus according to claim 5, further comprising:
means for generating color signals based on said at least one kind of B-mode image data generated by said image data generating means.

10. An ultrasonic imaging apparatus according to claim 1, wherein said evaluating means evaluates a spatial intensity distribution of said group of reception signals and/or statistics values calculated based on the spatial intensity distribution as said mutual property.

11. An ultrasonic imaging apparatus according to claim 1, wherein said evaluating means evaluates a degree of specular reflection components contained in said group of reception signals.

12. An ultrasonic imaging apparatus according to claim 1, wherein said evaluating means discriminates whether the predetermined region within the object is a hard tissue or a soft tissue based on said mutual property.

13. An ultrasonic imaging method of obtaining information for generating an ultrasonic image based on reception signals obtained by using an ultrasonic probe including plural ultrasonic transducers for transmitting ultrasonic waves toward an object to be inspected and receiving ultrasonic echoes propagating from the object to output reception signals, said method comprising the steps of:
(a) evaluating mutual property of a group of reception signals relating to a region within the object from among the reception signals respectively outputted from said plural ultrasonic transducers; and
(b) amplifying said group of reception signals with signal amplification factors determined with respect to respective reception signals based on an evaluation result at step (a).

14. An ultrasonic imaging method according to claim 13, wherein step (a) includes obtaining tissue property in a region, where the ultrasonic echoes propagating from the object are generated, based on said mutual property, and determining signal amplification factors of said group of reception signals with respect to respective reception signals according to the tissue property of said region.
